(19) United States
(12) Patent Application Publication
(10) Pub. No.: US 2004/0181193 A1
(43) Pub. Date: Sep. 16, 2004

(54) Guide and holding means

(76) Inventor: Fredrik Nilsson, Ekero (SE)

Correspondence Address: Rolf Fasth, Fasth Law Offices, 629 East Boca Raton, Phoenix, AZ, 85022, US

(21) Appl. No.: 10/485,475

(22) PCT Filed: Jul. 15, 2002
(86) PCT No.: PCT/SE02/01344

(51) Int. Cl. A61M 5/00

(52) U.S. Cl. 604/257

(57) ABSTRACT

Guide and holding means (1) for use in intestinal lavage by means of an enema device comprising an anal connector (4) connected to liquid tube (5). The means (1) is elongated and provided in its first end portion (2) with attachment means (3) for, at use, a utilized anal connector (4) and that the means (1) is intended to be supported by a rim of a toilet at a portion (9) of the means (1) positioned between the first end portion (2) and a second end portion (8). The first end portion (2) in a lever way is guideable and holdable in a desired position by hand force applied in the second end portion (8).

The present invention concerns guide and holding means for use at intestinal lavage by means of enema means comprising an anal connector connected to a liquid tube.

### BACKGROUND ART

Persons with faeces incontinence or repetitive constipations need to rinse the colon by means of the injection of an enema liquid in the colon. This is done by holding an anal connector connected to liquid tube against anus and/or inserting it into anus by means of the hand, whereafter enema liquid, for example tepid water, is injected into the colon. Generally, the enema liquid is present in a receptacle, which is positioned at a suitable level in order to achieve a suitable pressure and flow for the liquid into colon. A liquid lock of any kind is usually provided along the tube.

If the person is disabled, elderly or a child it is difficult to manage this on their own but someone has to help to hold the anal connector against anus. This may be uncomfortable both for the person in question and the person giving help.

In medical care the patient is laid on the side during injection of enema liquid into the colon. Thereafter the patient must be transported to a toilet. At this transportation leaks often arise which demand extensive cleaning both of the patient and the enema room.

This problem has prior been attempted to be solved for example by means of a means for intestinal lavage as described in WO 85/04331. The means is releasable attached to a toilet and comprises two attachment means and a anal connector protruding up from the toilet. In order to use this the person sits down on the toilet at exactly the right place so that the anal connector protrudes into anus. It is not always that easy to find the exact right position whereby the person easily may be injured in the lower abdomen. This means is also difficult to bring and to clean.

Another variant of the same type of solution is shown in WO 94/25087. This means comprises a “bridge” extending over the opening of a toilet. In the bridge a stiff guide tube displaceable mounted so that the elevated end of the stiff guide tube may be displaced to the right position at anus. Thereafter a liquid tube is displaced through the guide tube and out of its end and into anus.

Even if this reduce the risks of injuries there is still the risk that the persons sits down in a wrong way, for example too low, so that the stiff guide tube may injure the person. The end height of the guide tube also varies with the height of the sit ring of the toilet. With a modern thin, flat sit ring the end of the guide tube will be in a too high position. If the person in question is small, the person will not obtain even support from the sit ring but maybe only in the front, whereby further risks of wrong position will occur. This means is difficult to keep clean and to bring when travelling.

### SUMMARY OF THE INVENTION

An object of the present invention is to enable at first hand disabled, elderly and younger people and people with limited ability to move to in an easy and safe way carry out intestinal lavage on their own. Another object is that the invention shall be easy to keep clean and easy to bring for example when travelling.

According to the invention the objects are met by guide and holding means according to claim 1.

Thanks to the fact that the guide and holding means according to the invention is elongated and uses the lever principle the anal connector can easily be guided to the anus and be pressed against this with desired pressure which easily may be manouvered by the person in question by means of the force of the hand. There is neither needed that a large force is applied at the other end due to the lever function. The lever ratio may be chosen by chosing at which point along the means it is supported at the rim of the toilet.

Since the means in its simpliest form is an elongated body with attachment means for an anal connector it is very easy to keep clean and since it is free from attachment means for attachment to a toilet it is not bulky and thus easy to bring.

### SHORT DESCRIPTION OF THE DRAWINGS

FIG. 1 shows a guide and holding means according to the present invention together with a conventional enema device,

FIG. 2 shows theoretically the use of a guide and holding means in relation to a toilet.

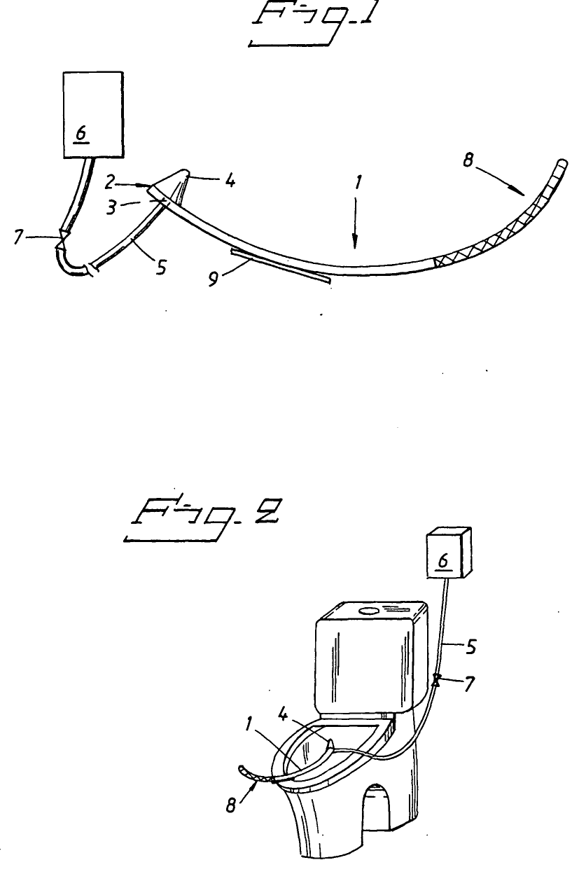

### DETAILED DESCRIPTION OF PREFERRED EMBODIMENTS

The guide and holding means 1 is elongated and preferably some what bended or arcuated to facilitate the handling and to give the right engagement direction against anus. In the first end portion 2 of the means 1 there is an attachment means 3. In the shown case it is a throughgoing hole but can be designed in another way depending on what is most suitable for the used conventional enema device.

For example an enema device called “Irrigator” from the American company Hollister may be used. It comprises a conical anal connector 4, a liquid tube 5 which is connectable to the anal connector 4 and a liquid receptacle 6. The liquid tube 5 is provided with a valve 7 for the closing and opening of the liquid tube 5.

The second end portion 8 of the means 1 may be formed as a handle, for example be provided with any grip enhancing material.

The portion of the means 1 which is aimed to be in contact with a rim of a toilet may be provided with a support portion 9. The support portion may for example be of rubber or provided with a rubber surface to decrease the risk of the means 1 sliding relative the rim of the toilet.

In use the anal connector 4 is attached to the means 1 and liquid is fed into the receptacle 6. The valve 7 is opened so that possible air in the liquid tube 5 is emptied, whereafter the valve 7 is closed again. The person sits down on the toilet and inserts the one end of the means 1 provided with the anal connector 4 between the legs and rests the means 1 against the rim of the toilet, whereafter the person maneuvers the other endportion 8 so that the anal connector comes into position whereafter the person applies a force in the other end so that the anal connector is pressed into anus. Thereafter the valve 7 is opened.

1. Guide and holding means (1) for use in intestinal lavage by means of an enema device comprising an anal connector (4) connected to a liquid tube (5), characterized in that the means (1) is elongated and provided in its first end portion (2) with attachment means (3) for, at use, a utilized anal connector (4) and that the means (1) is intended to be supported by a rim of a toilet at a portion (9) of the means (1) positioned between the first end portion (2) and a second end portion (8), whereby the first end portion (2) in a lever way is guideable and holdable in a desired position by hand force applied in the second end portion (8).

2. Guide and holding means according to claim 1, wherein the means is arcuate.

3. Guide and holding means according to claim 1 and 2, wherein the means (1) is provided with a separate support portion (9) for support against a rim of a toilet.

4. Guide and holding means according to claim 1, 2 or 3, wherein the second end portion (8) of the means is designed as a handle.

5. Guide and holding means according to any of the previous claims, wherein the attachment means (3) is a through hole.
